(19) United States
(12) Patent Application Publication
(10) Pub. No.: US 2011/0118765 A1
(43) Pub. Date: May 19, 2011

(54) ANASTOMOSIS STENT

(76) Inventor: Andres F. Aguirre, Chicago, IL (US)

(21) Appl. No.: 12/944,236

(22) Filed: Nov. 11, 2010

Related U.S. Application Data
(60) Provisional application No. 61/262,327, filed on Nov. 18, 2009.

(51) Int. Cl. A61B 17/11

(52) U.S. Cl. 606/153

(57) ABSTRACT

A stent made of single nitinol wire or other biocompatible material having shape memory properties may be configured to define a flower-like configuration generally providing a proximal plane and a distal plane, each generally centered around a generally cylindrical passage and configured for maintaining patency of an anastomosis or other opening. The wire or other biocompatible material may include a shroud or other covering and may also include one or more loops configured to allow relatively secure engagement of sutures. Methods of deployment are also provided.

### CROSS-REFERENCE TO RELATED APPLICATIONS

This application claims priority to U.S. Provisional Application Ser. No. 61/262,327, filed Nov. 18, 2009, and to U.S. patent application Ser. No. 12/620,864, filed Nov. 18, 2009, each of which is incorporated herein by reference in its entirety.

### TECHNICAL FIELD

The invention relates generally to medical stent devices. More particularly, the invention pertains to a stent configured to maintain patency of an anastomosis or other surgery-related aperture.

### BACKGROUND

Magnetic anastomosis devices (MADs) are currently used to create an anastomotic channel across two viscera separating portions of a body lumen (e.g., the alimentary canal and bodily systems feeding thereinto) for the purpose of redirecting bodily fluids. For example, intestinal contents or bile may be redirected in patients who have developed an obstruction of the bowel or bile duct due to such conditions as tumor, ulcer, inflammatory strictures, or trauma. A magnetic anastomosis device is disclosed in U.S. Pat. No. 5,690,656, the disclosure of which is incorporated herein by reference in its entirety. Generally, the MAD includes first and second magnet assemblies comprising magnetic cores that are surrounded by thin metal rims. Due to the magnetic attraction between the two magnetic cores, the walls of two adjacent viscera may be sandwiched and compressed between the magnet assemblies, resulting in ischemic necrosis of the walls to produce an anastomosis between the two viscera. The viscera treated by MADs include the gall bladder, the common bile duct, the stomach, the duodenum, and the jejunum of the small intestine.

An anastomosis created using MADs or other surgical means may be useful for facilitating a NOTES (natural orifice translumenal endoscopic surgery) procedure, whereby “scarless” abdominal operations can be performed with an endoscope passed through a natural orifice (mouth, urethra, anus, etc.) then through an internal incision in—for example—the stomach, vagina, bladder, or colon, thus avoiding any external incisions or scars. It is important that a translumenal anastomosis remain patent, whether it is configured for use to bypass other structures (e.g., bypassing a diseased or injured proximal portion of the duodenum by providing an aperture directly from the stomach lumen to a more distal portion of the duodenum) or for use in providing access for surgical devices, such as during a NOTES procedure. Other natural, but surgically enhanced openings (e.g., a cannulated Sphincter of Oddi) may also need to be kept open/patent to facilitate surgical access, drainage of fluid, etc.

Various tubular stents are known for maintaining patency of generally tubular passages such as blood vessels or bile ducts. However, such tubular devices are not well suited to be maintained effectively in the relatively short longitudinal length of a translumenal anastomosis (e.g., gastro-duodenal anastomosis) or for maintaining the open patency of a cannulated sphincter. Accordingly, it would be useful to provide a device configured to effectively maintain patency of such openings.

### BRIEF SUMMARY

In certain aspects, stent configurations of the present invention may provide patency for surgical anastomosis created by MADs or other means. A single nitinol wire or other biocompatible material having shape memory properties may be configured to define a flower-like configuration generally providing a proximal plane and a distal plane, with a generally cylindrical passage provided therebetween. The wire or other biocompatible material may include a shroud or other covering and may also include one or more loops configured to allow relatively secure engagement of sutures.

### BRIEF DESCRIPTION OF THE DRAWINGS

FIGS. 1A-1B show one embodiment of a stent device;

FIGS. 2A-2B show the stent device of FIGS. 1A-1B, deployed in tissue to maintain open patency of an anastomosis;

FIG. 3A shows another stent device embodiment, having fewer arches than the embodiment of FIGS. 1A-1B;

FIGS. 3B-3D show other stent device embodiments, each having a loop structure at the end of one or more arches;

FIGS. 4A-4B show, respectively, the stent embodiment of FIG. 3D from a top plan view including a suture, and from a side view with the suture actuated to compress one set of arches together;

FIGS. 5A-5C show one method of deploying a stent device in an anastomosis or other aperture;

FIG. 6 shows a multiband ligator device configured for deploying a stent device;

FIG. 7 shows another multiband ligator device configuration for deploying a stent device; and

FIGS. 8A-8B show a stent device with the interior regions of its arches being occupied by a fabric or other material.

### DETAILED DESCRIPTION

As defined herein, the term “proximal” refers to an end direction nearer a physician or other person administering a treatment or procedure, while the term “distal” refers to an end direction nearer a patient receiving the treatment or procedure, opposite the proximal end. The term “wire” refers to an elongate slender structure formed of a biocompatible shape memory material such as nitinol, another alloy, or an appropriate polymer; it may include an extruded fine, flexible rod-like structure, or may be laser-cut or otherwise formed from a sheet of material.

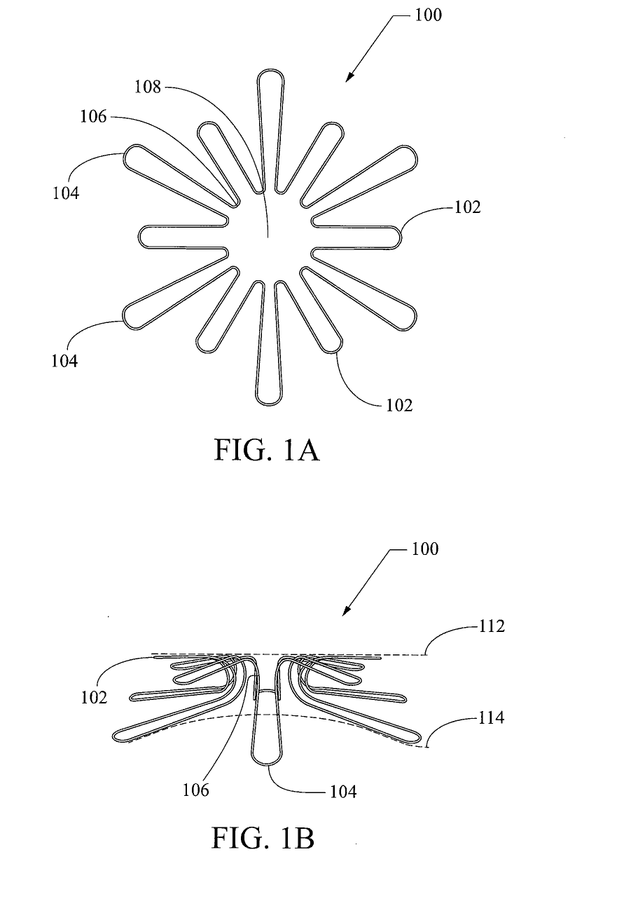

One embodiment of an anastomosis stent 100 is described with reference to FIGS. 1A-1B. In a preferred configuration, the stent 100 includes a single continuous unitary nitinol wire (e.g. pulled, extruded, laser-cut from a sheet) or another unitary structure formed from another elongate wire of biocompatible shape memory material. The continuous unitary wire structure may be seamless, or may have its ends welded, crimped or otherwise connected at a one or more points, including that a plurality of components may be used (e.g., in an end-to-end configuration). The stent 100 includes a flower-like configuration, shown in the top plan view of FIG. 1A. A plurality of alternating conjoined arches 102, 104 generally define, respectively, an upper plane 112 and a lower plane 114. As shown in the side view of FIG. 1B, the planes 112, 114 may be curved (e.g., so as to form, for example, a hemispherical or conoidal plane). It should be appreciated that the planes may also be generally flat or curved in a variety of other configurations, when the apical portions of the first and/or second sets arches 102, 104 are maximally separated from each other. The phrase “maximally separated from each other” is used herein to describe a configuration wherein the apical tip of each arch in a given plan is separated from each other arch in that plane by a maximum distance in view of the configuration of the arches and in view of a desired planar configuration such that it is not limited to a flat plane where the apices of the arches defining a plane are at an absolute maximum separation. Each curved plane may have a different contour than another curved plane. When using nitinol or another biocompatible shape memory material, it may be advantageous to heat-set or otherwise pre-form or pre-set the formed stent into a deployed configuration.

The upper arches 102 defining the upper plane 112 are shown as being shorter from a central longitudinal axis of the stent 100, relative to the lower arches, but it should be appreciated that the relative lengths of each arch may be varied to serve particular needs (e.g., to fit particular patient body structures) within the scope of the present invention. The conjoined arch bases 106 are themselves arches that are generally transverse to, and that form transitional regions between, each of the adjacent arches 102, 104 when the stent is deplored. Together, the apical portions of those transitional curved base arches 106 form a generally cylindrical central passage 108 (circular as shown, but including that the central passage may have an elliptical, obround, rectilinear, or other geometrically-configured cross-section around which all of the arches are generally centered). The generally circular central passage 108 formed thereby is configured to maintain open patency of an anastomosis, and preferably is about the same thickness as the anastomosis. In preferred embodiments, adjacent arches of each plane are connected to and continuous with arches of the other plan rather than with arches of the same plane. Also, it is preferable that, as viewed from a longitudinal end, the arches of each plane are offset from the arches of the other plane.

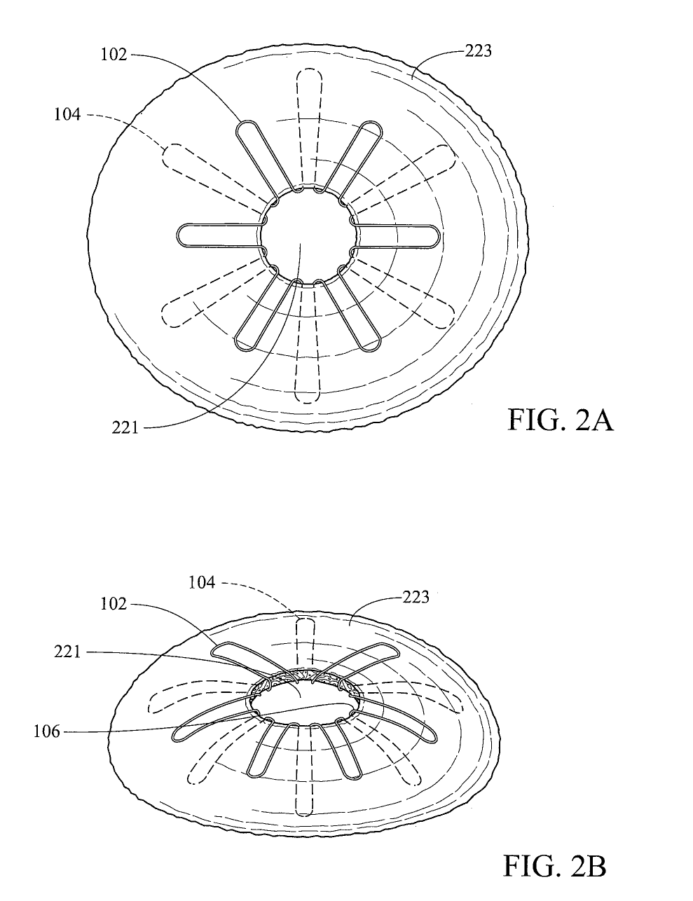

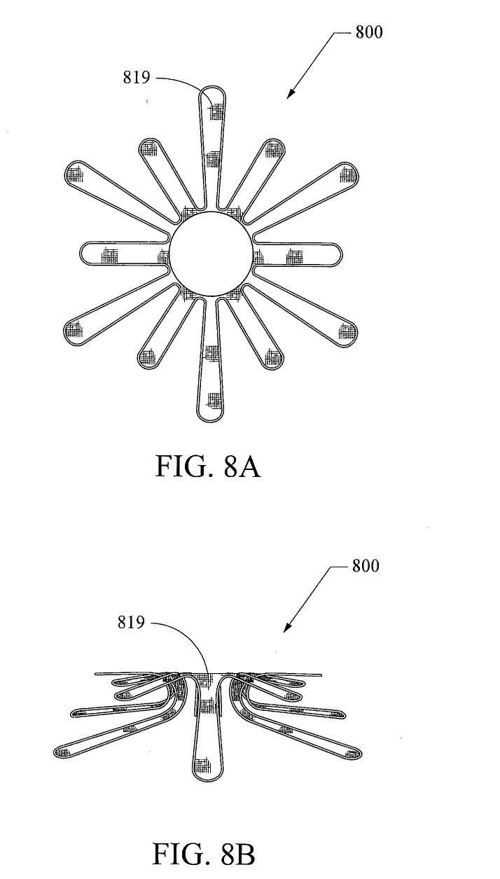

FIGS. 2A-2B show, respectively, top and top perspective views of a stent 100 disposed across an anastomosis 221 in a manner configured to keep the anastomotic opening 221 open. The anastomosis 221 is shown as an opening through tissue 223. The stent upper arches 102 are disposed on the proximal face of the tissue 223, and the stent lower arches 104 (shown in dashed lines) are disposed on the distal face of the tissue 223. The conjoined arch bases 106 retain the open configuration of the anastomosis 221. The disposition of the upper and lower stent arches 102, 104 preferably distributes surface forces generally evenly around the length of the stent wire to minimize likelihood of the stent causing damage to the tissue 223. It should be appreciated that a fabric or other covering may be provided over a portion or all of the stent, such as—for example—a silicone membrane that may be molded or otherwise applied across or between the arches. Such a construction would provide further distribution of surface contact forces on the tissue and may also help to minimize undesirable tissue in-growth. FIGS. 8A-8B show a stent 800 of the present invention, configured like the stent 100, except that it also includes a shroud or other covering 819 formed of a biocompatible fabric such as, for example, a polymeric fabric of the type known and used in vascular stent grafts. The covering is shown as just filling the inner portion of the arches, but it should be appreciated that a covering could be provided that would span spaces between two or more arches adjacent in one of the planes (e.g., using pleated or elastic material that would not interfere with deployment), or the space within at least one arch, up to and including a plurality of arches, and even all arches. That is, a covering may be provided across an inner portion of at least one of the first plurality of upper stent arches 102, one of the second plurality of lower stent arches 104, or any combination thereof, up to and including all of the arches. Alternatively or in addition, a covering may be provided between adjacent upper stent arches 102 and/or between lower stent arches 104, including any combination thereof.

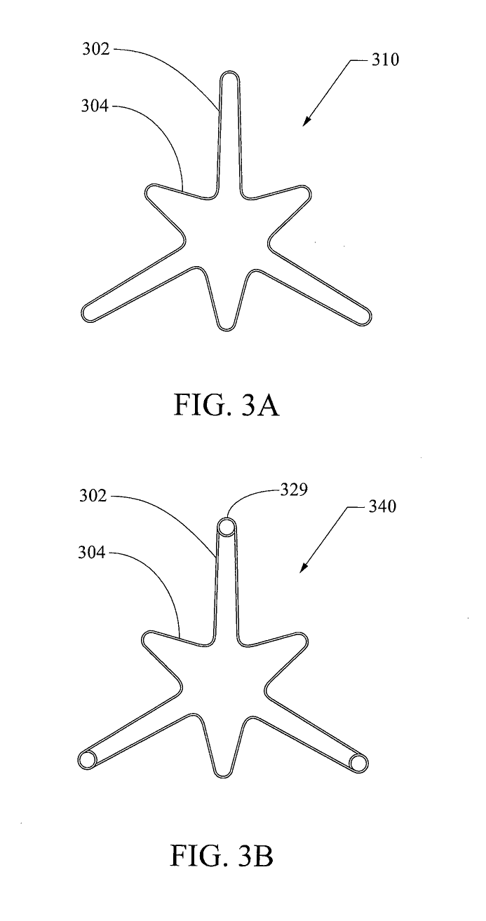

FIGS. 3A-3D show other stent embodiments, each of which includes fewer, less symmetrical arches than the embodiment of FIGS. 1A-1B. FIG. 3A shows a stent 310 with relatively long arches 302 and relatively short arches 304. This configuration may be placed with the longer arches 302 on a side of an opening best suited to prevent undesirable migration, and/or the shorter arches 304 may be oriented on a longitudinal opening side that is more constricted (e.g., within the base of the common bile duct, with the longer ends in the duodenum when using the stent to facilitate maintaining dilation of the cannulated Sphincter of Oddi). As shown in FIGS. 3A-3D, preferred embodiments generally may include a plurality of arches including at least three arches forming the upper and/or lower planes, although, as shown in FIGS. 1A-2B, each of the planes formed by the arches may include more than three arches.

FIGS. 3B-3D show loops 329 at the ends of the shorter and/or longer arches 304, 302. The loops 329 have several potential applications. In addition to, or instead of, serving as a means for maintaining a patent passage, stents of the present invention may serve to anchor other structures in or near an aperture such as an anastomosis, sphincter, or other opening. In either use, it may be advantageous to suture one or more of the arches to underlying tissue. The loops 329 may provide an anchoring point for a suture 327, such as is shown in FIG. 3C. The loop structure 329 will prevent the suture from migrating around the periphery of a stent 330, 340, particularly during placement (after which a suture placement device may be used to secure the suture to tissue).

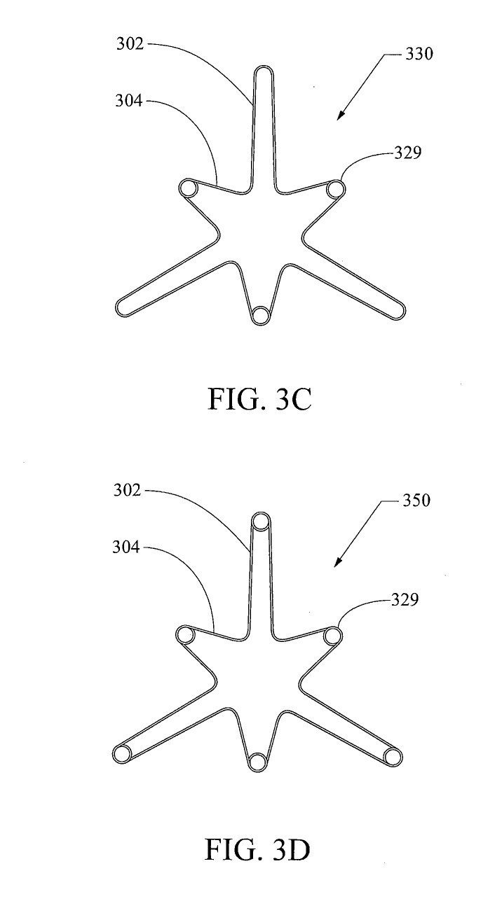

In another application, a suture 355 may be placed through the loops 329 in a drawstring manner. It will be appreciated that this configuration will allow one to radially collapse the arches toward each other during introduction and/or retrieval of the stent. FIGS. 4A-4B show one way in which a suture 355 directed through the loops 329 of the stent 350 (as in FIG. 3D). FIG. 4A shows an end view with the suture 355 directed through the loops 329 of the stent 350, where the longer arches 302 are deployed (i.e., spread out to a broader radius). As shown in the side view of FIG. 4B, a grasper 470 has been deployed, grasping and pulling the suture in drawstring fashion to radially collapse the arches 302 sufficiently that they can be withdrawn through the aperture in the tissue 475 where the stent 350 is deployed. Thus, the suture may be included with the device and operate as a circumferential constraining structure in mechanical communication with apical portions of at least one plurality of arches.

A preferred construction includes a single nitinol wire having its ends welded together. In the embodiments described herein, this construction will provide a low profile and high resistance to migration of the stent device. The planes formed by the upper and lower arches preferably are generally parallel, whether they are flat, curved, or partially spherical, but they spaced apart such that they do not intersect within the borders of the plane defined by apices of the arches. One exemplary embodiment described with reference to FIGS. 1A-1B may be configured to maintain a patent anastomosis between the stomach and duodenum of a patient. The longer arches 104 defining the plane 114 each extend about 40 mm from the central longitudinal axis of the device, and the shorter arches 102 defining the plane 112 each extend about 20 mm from the central longitudinal axis of the device 100. As illustrated, this exemplary embodiment will fit a 16 mm diameter anastomosis. Those of skill in the art will appreciate that the shapes and dimensions of the arches, the shapes and dimensions of the central opening, the thickness and rigidity of the wire body, and other traits of the device may be altered within the scope of the present invention to provide desirable stiffness, fittedness to an anastomosis or other opening, and other adaptations for use to maintain a patent opening (e.g., that may be elliptical, adjacent to softer or harder tissues, etc.). The number and relative proportions of the arches may be varied as well.

Biocompatible polymers having shape-memory characteristics, radio-opaque and/or echogenic markers, endoscopically-visualizable colors, or other features may also be used in stents constructed within the scope of the present invention. In addition to maintaining anastomoses created for NOTES or other procedures, stents of the present invention may be used hold two tissues together. For example, pancreatic pseudocysts may be treated by cystgastronomy, cystjejunostomy, or cystduodenostomy (respectively, forming a surgical connection between the pseudocyst and the stomach, jejunum, or duodenum) to facilitate drainage of the cyst. In each of the procedures described herein, a stent device as disclosed herein may be used temporarily or permanently to maintain patency of an opening.

Different methods of introduction may be used to facilitate placement of a stent of the present invention into an opening where it will be useful to maintain patency. Those of skill in the art will appreciate that the drawstring functionality described above with reference to FIGS. 4A-4B may be useful for reducing a stent into a low columnar profile/smaller radius configuration during an introduction procedure. Indeed that approach may be used in conjunction with other methods described below.

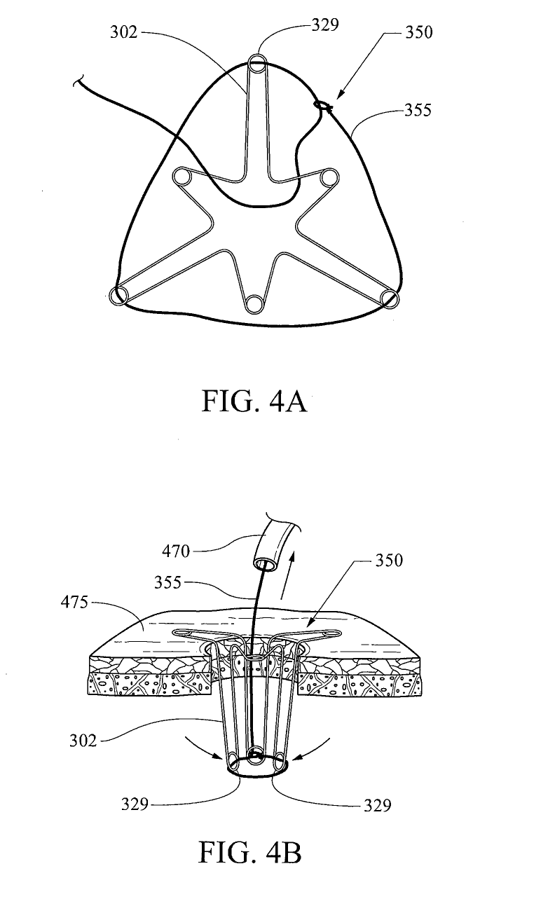

One method of introduction is described with reference to FIGS. 5A-5C. As shown in FIG. 5A, a stent 500 is provided in a columnarly-collapsed/restrained (that is, a non-deployed) configuration within the distal end of a delivery catheter 585 or other generally cylindrical delivery device, such that the apices of the lower and upper arches are each respectively drawn in close proximity with each other. This non-deployed configuration preferably is configured for substantially atraumatic passage through at least a portion of an anastomosis. The shape-memory bias of the stent 500 toward an outward/expanded configuration will keep it retained in the lumen of the catheter 585 (in all embodiments, it is preferable that the stent be biased into a deployed/expanded configuration by heat-set or other means using shape-memory or other appropriate materials). As shown in FIG. 5B, the distal end of the catheter 585 may be directed just through a pair of adjacent apertures between the stomach wall 590 and jejunal wall 592 (which aperture may have been formed and initially secured by MADs, not shown). A pusher 587 may be deployed through the catheter lumen to push the stent 500 just far enough distally that its distal arched arms 502 are allowed to assume their expanded larger radius configuration. Then, as shown in FIG. 5C, the catheter 585 can be withdrawn proximally, bringing the distal arched arms 502 into contact with the jejunal wall 592. That contact and/or additional distal motion from the pusher 587 will then deploy the proximal arched arms 504 of the stent 504, securing the gastric and jejunal walls 590, 592 together with a patent anastomotic aperture spanning them.

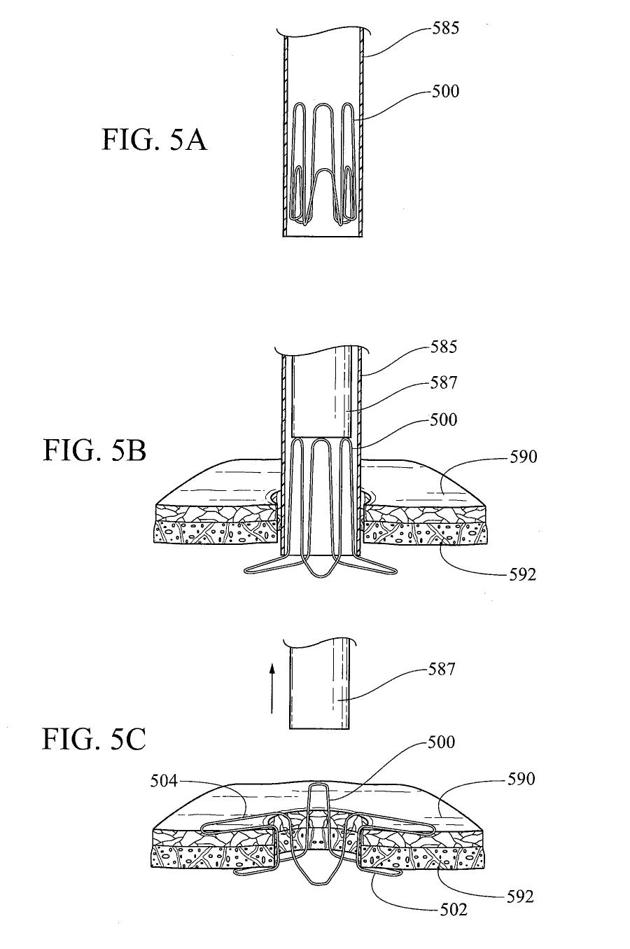

Another deployment method, illustrated with reference to FIG. 6, allows deployment of a stent 600 using a multiband ligator (MBL) cap 623 (of the type sold by Cook Endoscopy, Inc., Winston-Salem, N.C.; cf. U.S. Pat. Nos. 5,624,453 and 6,149,659, each of which is incorporated herein by reference). The cap 623 is mounted to the distal end of an endoscope 625. The stent 600 may be disposed within the cap 623 as shown in FIG. 6. Then, deployment may be effected using a pusher structure in much the same manner as described above with reference to FIGS. 5A-5C. As such, a catheter, MBL, or other cylindrical or tubular structure may be included with the device and operate as a circumferential constraining structure in mechanical communication with apical portions of at least one plurality of arches.

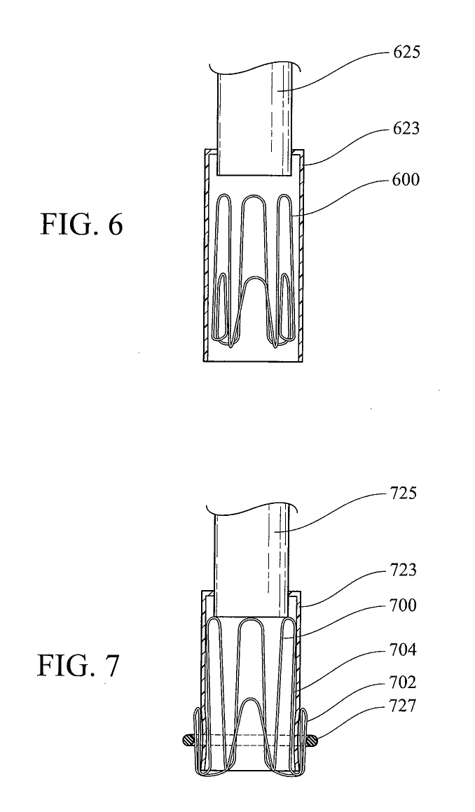

Another deployment method is illustrated with reference to FIG. 7, also using an MBL cap 723 mounted to the distal end of an endoscope 725. In this embodiment, the distal arches 702 of the stent 700 are collapsed and disposed on the exterior of the MBL cap 723 mounted to the distal end of an endoscope or other device 725, where they are secured in a radially low profile by an MBL band ring 727. The proximal arches 704 of the stent 700 may be disposed within the cap 723 as shown in FIG. 7. The distal end of the assembly may be directed through a target aperture, then deployment may be effected by releasing the band ring 727 in the manner known in the art, thereby freeing the distal arches 702, after which the cap 723 and scope 724 can be withdrawn to complete stent deployment in the same manner as described above with reference to FIGS. 5A-5C.

In each of these methods, at least a proximal portion of the stent is circumferentially constrained to present a radial profile that has a smaller outer diameter than when the arches are deployed. Specifically, in a fully deployed configuration, the outer ends of the arches will be at or near the furthest possible distance from each other allowed by the planar configuration they will occupy. However, when constrained for delivery/deployment or retraction, the apices of the arches will all be drawn near each other to present a lower-profile that can be directed through, for example, body passages, an endoscope working channel, or be mounted for delivery into a structure such as is described above. In all embodiments, it is most preferable that the expanded/deployed configuration be the configuration assumed when the shape-memory materials are released from constraint in a body space. As shown, for example, with reference to FIGS. 1A-1B and 3A-3B, an outer diameter/circumference formed by the apices of one set of arches along one plane (e.g., upper arches 102, 304) may be greater than or less than an outer diameter/circumference formed by the apices of the set of arches along the opposite plane (e.g., lower arches 104, 302), and the difference between those diameters/circumferences may be slight or significant.

Drawings in the figures illustrating various embodiments are not necessarily to scale. Some drawings may have certain details magnified for emphasis, and any different number or proportions of parts should not be read as limiting, unless so-designated by one or more claims. Those of skill in the art will appreciate that embodiments not expressly illustrated herein may be practiced within the scope of the present invention, including that features described herein for different embodiments may be combined with each other and/or with currently-known or future-developed technologies while remaining within the scope of the claims presented here. It is therefore intended that the foregoing detailed description be regarded as illustrative rather than limiting. And, it should be understood that the following claims, including all equivalents, are intended to define the spirit and scope of this invention.

I claim:

1. A medical stent device, comprising:
a biocompatible shape memory material formed as a continuous unitary wire structure including a first plurality of arches defining a first plane, a second plurality of arches defining a second plane spaced apart from the first plane, and a third plurality of arches connecting the first and second plurality of arches;
wherein the third plurality of arches is generally transverse relative to the first and second plurality of arches, and apical portions of the third plurality of arches define a generally cylindrical opening around which the first, second, and third pluralities of arches are generally centered.

2. The device of claim 1, further comprising a multiband ligator cap.

3. The device of claim 1, comprising a deployment configuration wherein apical portions of the first plurality of arches, opposite the third plurality of arches, are unconstrained within a generally cylindrical space having a second diameter that is less than a first diameter defined by the apical portions of the second plurality of arches when said apical portions of the first plurality of arches are maximally separated from each other.

4. The device of claim 3, wherein the biocompatible shape memory material comprises a pre-set shape configured with the apical portions of the first plurality of arches maximally separated from each other.

5. The device of claim 3, wherein the first diameter defined by the first plurality of arches when said apical portions of the first plurality of arches are maximally separated from each other is less than a third diameter defined by the second plurality of arches when said apical portions of the second plurality of arches are maximally separated from each other.

6. The device of claim 4, wherein the first plane is formed when the apical portions of the first plurality of arches are maximally separated from each other, and the first plane comprises a curved plane.

7. The device of claim 1, further comprising a covering across an inner portion of at least one of the first plurality of arches, one of the second plurality of arches, or any combination thereof.

8. The device of claim 7, wherein the covering comprises a biocompatible fabric.

9. The device of claim 1, further comprising a covering between adjacent ones of the first plurality of arches, adjacent ones of the second plurality of arches, or any combination thereof.

10. The device of claim 1, wherein the first plurality of arches includes at least three arches.

11. The device of claim 1, further comprising a circumferential constraining structure configured to constrain at least one of the plurality of arches in a diameter less than a maximally expanded diameter.

12. The device of claim 11, wherein the circumferential constraining structure comprises a suture in mechanical communication with apical portions of at least one plurality of arches.

13. The device of claim 11, wherein the circumferential constraining structure comprises a tubular structure in mechanical communication with apical portions of at least one plurality of arches.

14. The device of claim 1, wherein the first plane comprises a curved plane.

15. A method for deploying a medical stent device, the method comprising the steps of:
providing a medical stent device according to claim 1;
disposing at least a proximal portion of the medical stent device within a generally cylindrical delivery device such that the first plurality of arches is circumferentially constrained together to present a radial profile smaller than when the arches are allowed to separate from one another;
circumferentially constraining the second plurality of arches;
directing the generally cylindrical delivery device and medical stent device adjacent a target site comprising an aperture therethrough;
directing the second plurality of arches to one face of the aperture, said aperture having a diameter less than an expanded diameter of each of the first and second plurality of arches' apices and a thickness of about the same as a longitudinal thickness of the generally cylindrical opening; and
releasing the constraint of one of the first or second plurality of arches, allowing the arches to deploy.

16. A medical stent device, comprising:
a biocompatible shape memory material formed as a continuous unitary wire structure including a plurality of alternating first arches and second arches, and having a first, non-deployed configuration and a second, deployed configuration;
wherein, the first, non-deployed configuration includes the first plurality of arches having their apices all drawn in proximity to each other, thereby forming a generally columnar first low profile and the second plurality of arches having their apices all drawn in proximity to each other, thereby forming a generally columnar second low profile;
wherein, the second, deployed configuration includes the first plurality of arches having their apices spread apart each other, the first arches' apices thereby forming a generally columnar first expanded profile having an outside diameter greater than the first low profile, and the second plurality of arches having their apices spread apart each other, the second arches' apices thereby forming a generally columnar second expanded profile having an outside diameter greater than the second low profile;
wherein, in the second deployed configuration, the first plurality of arches defines a first plane, the second plurality of arches defines a second plane that is spaced apart from the first plane; and
wherein apices of a third plurality of arches connect the first and second plurality of arches, the apical portions of the third plurality of arches define a generally cylindrical opening around which the first, second, and third pluralities of arches are generally centered, and the apical portions of the third plurality of arches are generally transverse relative to the first and second plurality of arches in the second, deployed configuration.

17. The device of claim 16, wherein the first plane comprises a curved plane.

18. The device of claim 16, wherein the second plane comprises a curved plane having a different contour than the first plane.

19. The device of claim 16, wherein the generally cylindrical opening defined by the apical portions of the third plurality of arches is configured to maintain open patency of an anastomosis.

20. The device of claim 16, wherein the first, non-deployed configuration is configured for substantially atraumatic passage through at least a portion of an anastomosis.
